(19) United States
(12) Patent Application Publication
(10) Pub. No.: US 2024/0277682 A1
(43) Pub. Date: Aug. 22, 2024

(54) PHARMACEUTICAL HYDRONIDONE FORMULATIONS FOR DISEASES

(71) Applicant: Gyre Therapeutics, Inc., San Diego, CA (US)

(72) Inventors: Ying LUO, Shanghai (CN); Ling ZHANG, Beijing (CN)

(21) Appl. No.: 18/381,550

(22) Filed: Oct. 18, 2023

Related U.S. Application Data
(63) Continuation of application No. PCT/CN2021/088104, filed on Apr. 19, 2021.

(51) Int. Cl. A61K 31/4418; A61P 1/16

(52) U.S. Cl. CPC A61K 31/4418; A61P 1/16

(57) ABSTRACT

It relates to a method of treating a subject with liver fibrosis and/or liver cirrhosis. The disclosure provides a pharmaceutical composition that includes hydronidone or a salt thereof that can be used for treating a subject with liver fibrosis and/or liver cirrhosis. Liver stiffness measurements values and/or Ishak scores may be used to identify subjects for treatment of liver fibrosis and/or liver cirrhosis.

### BACKGROUND

Liver fibrosis is a life-threatening disease with high morbidity and mortality. Currently, there is no FDA-approved treatment of liver fibrosis in the US and many parts of the world are in need of better treatments.

### SUMMARY

In an aspect, the present disclosure provides a method of treating a subject with liver fibrosis comprising administering to the subject a pharmaceutical composition comprising hydronidone or a salt thereof.

In another aspect, the present disclosure provides a method of treating a subject with liver cirrhosis comprising administering to the subject a pharmaceutical composition comprising hydronidone or a salt thereof.

In another aspect, the present disclosure provides a method of treating a subject with advanced hepatitis B viral infection comprising administering to the subject a pharmaceutical composition comprising hydronidone or a salt thereof.

In another aspect, the present disclosure provides a method of treating a subject with NASH fibrosis comprising administering to the subject a pharmaceutical composition comprising hydronidone or a salt thereof.

In another aspect, the present disclosure provides a method of treating a subject comprising identifying a subject having a liver stiffness measurement value of at least 4 kiloPascals (kPa) and administering to the subject a pharmaceutical composition comprising hydronidone or a salt thereof.

In another aspect, the present disclosure provides a method of treating a subject comprising identifying a subject with an Ishak value that is from about 1 to 6; and administering to the subject a pharmaceutical composition comprising hydronidone or a salt thereof and a pharmaceutically acceptable excipient.

In another aspect, the present disclosure provides a method of treating a subject comprising identifying a subject that is substantially free of hepatitis B viral protein; and administering to a subject a pharmaceutical composition comprising hydronidone or salt thereof, and a pharmaceutically acceptable excipient.

In another aspect, the present disclosure provides a method of treating a subject comprising identifying a subject wherein the subject tests positive for hepatitis virus DNA after receiving one or more courses of anti-viral treatment; and administering to the subject a pharmaceutical composition comprising hydronidone or a salt thereof and a pharmaceutically acceptable excipient.

In another aspect, the present disclosure provides a pharmaceutical composition comprising about 20% to about 90% weight percentage of hydronidone or a salt thereof and one or more pharmaceutically acceptable excipients. In some cases, the weight percentage of hydronidone or a salt thereof is about 25% to about 35%. In some cases, the one or more pharmaceutically acceptable excipients are selected from lactose, sucrose, magnesium stearate, glucose, plant cellulose, calcium carbonate, zinc stearate, calcium stearate, stearic acid, palmitic acid, myristic acid, glyceryl dibehenate, and talc.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows ishak score analysis of various drug groups.

FIG. 2 shows analysis of intra group ishak score change.

FIG. 3 shows analysis of patients with significant fibrosis.

FIG. 4 shows combination analysis of 270 mg and 360 mg groups.

FIG. 5 shows combination of various drug groups.

FIG. 6 shows ishak score changes of baseline HBeAg(+) patients after 52 weeks.

FIG. 7 shows ishak score changes of baseline HBeAG(−) patients after 52 weeks.

FIG. 8 shows change of liver stiffness measurement (LSM).

FIG. 9 shows combination of drug groups for LSM analysis.

FIG. 10 shows analysis of HBV DNA (+) patients after 52 weeks of entecavir therapy.

FIG. 11 shows the effect of hydronidone on an induced NASH model in mice.

FIG. 12 shows H&E stains for hydronidone on an induced NASH model in mice.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “at most”, “less than,” or “less than or equal to” precedes the first numerical value in a series of two or more numerical values, the term “no more than,” “at most”, “less than,” or “less than or equal to” applies to each of the numerical values in that series of numerical values. For example, less than or equal to 3, 2, or 1 is equivalent to less than or equal to 3, less than or equal to 2, or less than or equal to 1.

### Overview

Liver cirrhosis or hepatic cirrhosis is a condition in which the liver does not function properly due to long-term damage. Scar tissue formation, known as fibrosis, is formed during the process of liver tissue repair. The presence of significant fibrosis from chronic liver damage and subsequent repair over time can lead to cirrhosis. Liver fibrosis is typically the first stage of liver scarring. A number of factors may result in hepatic fibrosis of the liver, for example, any process that may cause damage to liver homoeostasis (e.g., inflammation, toxic damage, change in hepatic blood flow and liver infection (virus, bacteria, fungi and parasites), etc.).

The anti-fibrotic effects of hydronidone were observed in in-vivo models of liver fibrosis of different etiology described herein. Hydronidone anti-fibrotic effects were evaluated in several animal models of liver fibrosis, such as human serum albumin (HSA)-induced liver fibrosis in rats, dimethyl nitrosamine (DMN)-induced liver fibrosis in rats, and carbon tetrachloride (CCl₄)-induced liver fibrosis in mice. Across these animal models, hydronidone exhibited potent anti-fibrotic effect, for example, decreasing significantly the level of fibrosis and improving the biochemical and pathological indices of liver fibrosis such as hydroxyproline content and levels of liver enzymes such as alanine aminotransferase (ALT) and aspartate aminotransferase (AST). In the CCl₄-induced liver fibrosis in mice, Hydronidone effects were the most prominent at the doses of 3-10 mg/kg/day, although some effects were also shown at the dose of 1 mg/kg/day. In some embodiments, this dose range corresponds to human equivalent dose (HED) range of 15-50 mg. In some embodiments, these doses are 7-24-fold lower than the human maximum daily dose of 360 mg (120 mg three times per day [TID]), administered to subjects with liver fibrosis due to chronic hepatitis B infection. In rats, depending on the model, anti-fibrotic effects of Hydronidone were shown in the dose range of 10-250 mg/kg/day, being comparable or more potent than silymarin extract, a known hepatic protectant, which was used as a positive control. The HED of these rat doses are in the range of 100-2,400 mg which were all tested as safe in Phase I clinical trials of Hydronidone, with the 100 mg dose being about one third less than the maximum daily dose of 360 mg (administered as 120 mg TID) administered to the subjects with liver fibrosis due to chronic Hepatitis B in the ongoing Phase II clinical study of efficacy and safety of Hydronidone in China.

### Methods of Treating

The present disclosure provides a method of treating a subject with liver fibrosis. The method may include administering hydronidone to a subject in need thereof. The method may include administering to the subject a pharmaceutical composition comprising hydronidone or a salt thereof and a pharmaceutically acceptable excipient.

The present disclosure also provides a method of treating a subject with liver cirrhosis. The method may include administering hydronidone to a subject in need thereof. The method may include administering to the subject a pharmaceutical composition comprising hydronidone or a salt thereof and a pharmaceutically acceptable excipient.

The present disclosure also provides a method of treating a subject with NASH fibrosis. The method may include administering hydronidone or a salt thereof to a subject in need thereof. The method may include administering to the subject a pharmaceutical composition comprising hydronidone or a salt thereof and a pharmaceutically acceptable excipient.

The present disclosure also provides a method of treating a subject with advanced hepatitis B viral infection. The method may include administering a pharmaceutical composition comprising hydronidone or a salt to the subject.

The present disclosure also provides a method of treating a subject comprising identifying a subject having a liver stiffness measurement value and administering to the subject a pharmaceutical composition comprising hydronidone or a salt thereof. The pharmaceutical composition comprising hydronidone or a salt thereof may be as described elsewhere herein. In some cases, the liver stiffness measurement value made be obtained by transient elastography. In some cases, the liver stiffness measurement value made be obtained by FibroTouch and/or FibroScan. In some cases, the liver stiffness measurement value may be calibrated to a reference standard. The reference standard may be, for example, a liver biopsy. The liver stiffness measurement value may be from about 4 kilopascals (kPa) to 13 kPa. The liver stiffness measurement value may be from about 4 kPa to 8 kPa. The liver stiffness measurement value may be from about 8 kPa to 13 kPa. The liver stiffness measurement value may be from about 8 kPa to 75 kPa. The liver stiffness measurement value may be at most about 13 kPa. The liver stiffness measurement value may be at most about 75 kPa.

The present disclosure also provides a method of a treating a subject comprising identifying a subject with an Ishak value and administering to the subject a pharmaceutical composition comprising hydronidone or a salt thereof. The pharmaceutical composition comprising hydronidone or a salt thereof may be as described elsewhere herein. The Ishak value may be from about 1 to 6. The Ishak value may be from 1 to 3. The Ishak value may be from 3 to 6.

In some embodiments, an Ishak score of 0 may indicate no fibrosis. In some embodiments, an Ishak score of 1 may indicate Fibrous expansion of some portal areas, with or without short fibrous septa. In some embodiments, an Ishak score of 2 may indicate Fibrous expansion of most portal areas, with or without short fibrous septa. In some embodiments, an Ishak score of 3 may indicate Fibrous expansion of most portal areas with occasional portal to portal bridging. In some embodiments, an Ishak score of 4 may indicate Fibrous expansion of portal areas with marked bridging (e.g., portal to portal as well as portal to central). In some embodiments, an Ishak score of 5 may indicate marked bridging (e.g., portal-portal and/or portal-central) with occasional nodules (e.g., incomplete cirrhosis). In some embodiments, an Ishak score of 6 may indicate cirrhosis, probable or definite.

In some embodiments, the Ishak value may be converted into another scoring system. The scoring system can be, for example, METAVIR scores, Knodell scores, etc.

In some embodiments, a method of a treating a subject may comprise identifying a subject with an METAVIR score and administering to the subject a pharmaceutical composition comprising hydronidone or a salt thereof. In some cases, a METAVIR score of F0 may indicate no fibrosis. In some cases, a METAVIR score of F1 may indicate portal fibrosis without septa. In some cases, a METAVIR score of F2 may indicate portal fibrosis with rare septa. In some cases, a METAVIR score of F3 may indicate numerous septa without cirrhosis. In some cases, a METAVIR score of F4 may indicate cirrhosis. In some cases, the METAVIR score may be from F0 to F4, F1 to F4, F2 to F4, F3 to F4, F1 to F3, F1 to F2, or F2 to F3.

The present disclosure also provides a method for treating a subject comprising identifying a subject that is substantially free of hepatitis B viral protein and administering to a subject a pharmaceutical composition comprising hydronidone or salt thereof.

The present disclosure also provides a method for treating a subject comprising identifying a subject wherein the subject tests positive for hepatitis virus DNA after receiving one or more courses of anti-viral treatment; and administering to the subject a pharmaceutical composition comprising hydronidone or a salt thereof. In some cases, the hepatitis virus DNA is selected from hepatitis A virus, hepatitis B virus, hepatitis C virus, hepatitis D virus, and hepatitis E virus. In some cases, the hepatitis virus DNA is hepatitis B.

### Pharmaceutical Compositions

The present disclosure provides a pharmaceutical composition. The pharmaceutical composition may include a compound shown in Formula (I)

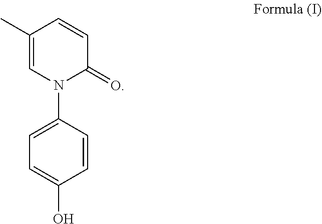

The compound of Formula (I) may be referred to as hydronidone and/or N-(4-hydroxyphenyl)-5-Methyl-2-pyridone. Hydronidone or a pharmaceutical composition described herein may be used for the treatment of liver fibrosis associated with chronic liver disease. Hydronidone or a pharmaceutical composition described herein may be used for the treatment of liver cirrhosis associated with chronic liver disease.

The pharmaceutical composition may comprise hydronidone. The pharmaceutical composition may include a weight percentage of hydronidone or a salt thereof. The weight percentage of hydronidone or a salt thereof in the pharmaceutical composition may be about 10% to 90%, 10% to 50%, 10% to 40%, 10% to 30%, 20% to 90%, 20% to 50%, 20% to 40%, 20% to 30%, 30% to 90%, 30% to 50%, or 30% to 40%. The weight percentage of hydronidone in the pharmaceutical composition may be at least about 20%, 25%, 30%, 35%, or 40%. The weight percentage of hydronidone in the pharmaceutical composition may be at most about 40%, 35%, 30%, 25%, or 20%. The weight percentage of hydronidone in the pharmaceutical composition may be about 30%.

The pharmaceutical composition may comprise hydronidone and one or more excipients. The one or more excipients may include, for example, lactose, sucrose, glucose, plant cellulose, calcium carbonate, magnesium stearate, zinc stearate, calcium stearate, stearic acid, palmitic acid, myristic acid, glyceryl dibehenate, and talc, etc. The one or more excipients may include magnesium stearate and lactose.

The one or more excipients may be a weight percentage of the pharmaceutical composition. The one or more excipients may be, for example, lactose. The weight percentage lactose in the pharmaceutical composition may be about 10% to 90%, 50% to 90%, 60% to 90%, 50% to 80%, 50% to 80%, 60% to 80%, 65% to 75%, 67% to 72%, or 68% to 71%. The weight percentage of lactose in the pharmaceutical composition may be at least about 50%, 60%, 65%, 67%, 68%, 69%, 70%, 71%, 72%, 73%, 75%, or 80%. The weight percentage of lactose in the pharmaceutical composition may be at most about 80%, 75%, 73%, 72%, 71%, 69%, 68%, 67%, 65%, 60%, or 50%. The weight percentage of lactose in the pharmaceutical formulation may be about 70%.

The one or more excipients may be, for example, magnesium stearate. The weight percentage of magnesium stearate in the pharmaceutical composition may be about 0.01% to 2%, 0.1% to 1.5%, 0.2% to 1.0%, 0.25% to 0.5%, 0.15% to 0.30%, 0.2% to 0.3%, or 0.21% to 0.29%. The weight percentage of magnesium stearate in the pharmaceutical composition may be at least about 0.01%, 0.05%, 0.1%, 0.15%, 0.20%, 0.25%, 0.30%, 0.35%, or 0.40%. The weight percentage of the one or more lubricating agents in the pharmaceutical composition may be at most about 0.40%, 0.35%, 0.30%, 0.25%, 0.20%, 0.15%, 0.10%, 0.05%, or 0.01%.

The pharmaceutical composition may comprise hydronidone, lactose, and magnesium stearate. The weight percentage of hydronidone, lactose, and magnesium may be about 30%, 69.8%, 0.2% respectively. The weight percentage of hydronidone, lactose, and magnesium may be about 30%, 69.7%, 0.3% respectively.

Provided herein, in certain embodiments, are compositions comprising a therapeutically effective amount of any compound or salt of hydronidone (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, and pastes for application to the tongue.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

A pharmaceutical composition, e.g., for oral administration, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the pharmaceutical agent. The matrices can be formed by solvent evaporation, spray drying, solvent extraction and other methods known to those skilled in the art.

Kits with unit doses of one or more of the agents described herein, usually in oral or injectable doses, are provided. Such kits may include a container containing the unit dose, an informational package insert describing the use and attendant benefits of the drugs in treating disease, and optionally an appliance or device for delivery of the composition.

### EXAMPLES

### Example 1: In Vitro Human Metabolism of Hydronidone Phase 1

The in vitro Phase I metabolism of hydronidone was evaluated by incubating human liver microsomes with hydronidone for 60 minutes. The sensitivity of the assay was demonstrated by incubating the human liver microsomes with dextromethorphan (positive control) for 20 min resulting in generation of metabolites dextrorphan and 3-methoxymorphinan. After the 60 min incubation of hydronidone in the human liver microsomes reaction system, no formation of metabolites was observed, indicating that the metabolic reaction in which hydronidone generated the M2 metabolite was not mediated by CYP450.

### Example 2: In Vitro Human Metabolism of Hydronidone Phase 2

The generation of the M4 metabolite was detected after the incubation of Hydronidone in the reaction system of human liver microsomes for 30 minutes, indicating that the metabolic reaction in which Hydronidone generated M4 is most likely mediated by uridine 5′-diphospho-glucuronosyltransferase (UDP-glucuronosyltransferase, UGT).

### Example 3: In Vitro Human Metabolism of Hydronidone CYP450 Interactions

In vitro study investigated whether Hydronidone is inhibitor for human cytochrome P450 (CYP450). The effects of different Hydronidone concentrations were determined by the fluorescence intensity of metabolic substrates of CYP1A2, 2C9, 2C19, 2 D6 and 3A4, produced under the effect of the relevant enzyme. The respective IC₅₀ value were calculated in the event of observed inhibition with hydronidone. Known CYP450 inhibitors were used as a positive control to demonstrate the sensitivity of the assay. The results showed no significant in vitro inhibitory activity of hydronidone on human CYP 1A2 (IC50 value>100 μM vs. positive control furafylline with IC50 value of 2.59 μM), 2C9 (IC50 value>100 μM positive control sulfaphenazole with IC50 of 0.49 μM), 2C19 (IC50 value>100 μM positive control tranylcypromine with IC₅₀ of 4.9 μM), 2D36 (IC50 value>100 μM positive control quinidine with IC50 of 0.01 μM), and 3A4 enzymes (IC50 value>100 μM vs. positive control ketoconazole with IC50 value of 0.027 μM). It can be concluded that Hydronidone is devoid of significant interactions with human CYP450.

### Example 4: In Vitro Human Metabolism of Hydronidone Pharmacokinetic Drug-Drug Interactions

The potential of hydronidone for drug interactions with two anti-viral drugs, Entecavir and Lamivudine, that are relevant to the indication of chronic hepatitis B infection in China, was evaluated in vitro using human liver microsomes. Entecavir is used concomitantly with Hydronidone in the ongoing Phase II trial of efficacy is safety in subjects with liver fibrosis due to chronic hepatitis B infection, in China. Entecavir and Lamivudine, were used concomitantly with Hydronidone, or individually, in the in vitro drug interaction experiments, in respective concentrations of each of 1.5 mM. The results obtained in the drug interaction studies indicated that: a) the liver metabolism is not the main Hydronidone metabolic pathway, b) entecavir and lamivudine do not exhibit drug interactions with Hydronidone as there were no significant difference of residual drug concentrations between individual medication groups and combined medication group (P>0.05).

### Example 5: Phase I Clinical Pharmacology Studies of Hydronidone Summary

| | | Test Product(s); | | |
|---|---|---|---|---|
| | | Dosage | | |
| | | Regimen; | | |
| Study | | Route of | Number of | Duration of |
| Identifier | Study Design | Administration | Subjects | Treatment |
| Example 7 | Randomized, open- | Hydronidone 200 mg | 12 subjects total, 3 | Single oral dose |
| | label, 4-period, | capsule, batch number: | subjects/group | |
| | cross-over, single- | 0711001. | | |
| | dose | Single oral doses of 200 | | |
| | | mg. 400 mg, 600 mg, and | | |
| | | 800 mg of Hydronidone | | |
| Example 8 | Randomized, open- | Hydronidone 200 mg | 12 subjects | Single oral dose |
| | label, 2-period, | capsule, batch number: | | |
| | cross-over, single | 0711001. | | |
| | dose, in presence | Single oral dose of | | |
| | and absence of food | 3 × 200 mg (total of 600 | | |
| | | mg) of Hydronidone | | |
| Example 9 | Randomized, open- | Hydronidone 200 mg | 27 subjects, 3 | Multiple oral |
| | label, parallel group, | capsule, batch number: | cohorts, 9 | doses |
| | multiple-dose | 071101. | subjects/group | administered |
| | | | | T.I.D., for 4 |
| | | | | consecutive days |
| Example 10 | Randomized, open- | Hydronidone 15 mg | A total of 52 | Single dose |
| | label, single- and | capsule, batch | subjects. | Multiple T.I.D. |
| | multiple- dose | number 20120602. | Part 1: 6-8 | dose for 7 days |
| | | Part 1: Single oral doses | subjects/cohort | and 4 weeks. |
| | | of 30 mg. 60 mg, 90 mg, | Part II: 6 | |
| | | and 120 mg of | subjects/cohort | |
| | | Hydronidone Part II: 90 | Part III: 6 | |
| | | mg and 120 mg oral dose | subjects/cohort | |
| | | administered T.I.D (270 | | |
| | | mg and 360 mg total daily | | |
| | | dose, respectively), for 7 | | |
| | | consecutive days. | | |
| | | Part III: 60 mg T.I.D. | | |
| | | (total of 180 mg/day), for | | |
| | | 4 weeks. | | |

### Example 6: Double-Blind Placebo-Controlled, In-Patient Study of Single and Multiple Doses of Hydronidone

The main objective of this study was to evaluate the tolerability of single and multiple ascending doses of Hydronidone in healthy subjects to support further Phase 1 studies as well to provide supportive information for dosage regimen in Phase II clinical trials.

This was a randomized, double-blind, placebo-controlled, in-patient study of single and multiple escalating doses of Hydronidone in healthy Chinese male and female subjects, 18-45 years of age, satisfying all inclusion and exclusion criteria. Hydronidone 100 mg and 200 mg capsule dosage strengths were used in the study. In every cohort, the subjects were randomly assigned to Hydronidone or placebo at a ratio of 3:1.

The single-dose component of the trial included 30 subjects that received 100 mg, 200 mg, 400 mg, 600 mg and 800 mg of Hydronidone, respectively (6 subjects/cohort). The post-dose observations were performed 24 hrs following the dosing. All subjects have completed the trial. Hydronidone was safe and well tolerated at all doses tested. No deaths nor serious adverse events (SAEs) were reported during the trial. Mild transient dizziness following administration of the test article was reported in one subject in each of the 800 mg Hydronidone and placebo cohorts. The dizziness resolved on its own, without sequelae. The post-dose values of some of the laboratory parameters were abnormal, both in Hydronidone and placebo groups. However, these changes were not considered clinically significant.

The multiple-dose component of the trial included 20 subjects assigned to receive 600 mg (8 subjects) three times per day (TID) (total daily dose of 1800 mg), or 800 mg of Hydronidone (12 subjects) TID (total daily dose of 2400 mg) for ten (10) consecutive days. Due to several cases of AEs of hypertriglyceridemia (4 out of 8 subjects) observed in the Hydronidone 600 mg TID dose group, the subjects (n=8) in the Hydronidone TID 800 mg dose group were divided into two subgroups, each comprising of 4 subjects. A staggered enrollment approach was applied: if no SAEs were observed in the first sub-group, then the enrollment of the subjects in the second sub-group could proceed with the same dose of Hydronidone 800 mg TID. If SAEs were observed in the first sub-group, then the Principal Investigator was to make further analysis to decide the dose level for the second sub-group. Due to inadvertent omission of the blood glucose test in the first sub-group (all four subjects) of the Hydronidone 800 mg TID dose group at the final study visit, per Principal Investigator decision, the enrollment was extended to include new four (4) subjects, increasing the total number of subjects in this dose group to 12. The safety evaluations were performed pre-dose, on the Day 5 of dosing, and 24 hr after the last dose of the 10-day dosing period.

No deaths nor SAEs were reported in this part of the trial. All subjects in the 600 mg TID cohort completed the study. In the 800 mg TID cohort all subjects completed the trial except one subject who was prematurely discontinued from the study on Day 6 based upon Investigator's perceived risks due to abnormal liver function parameters. This subject was assigned to Hydronidone treatment. In the 600 mg TID cohort, there were seven (7) AEs reported: 3 instances of hypertriglyceridemia, 1 instance each of chest tightness, abdominal distention, skin rash, and mouth ulcers; in the placebo group, one instance of rash and one instance of hyperlipidemia were reported. The increased lipid parameters (triglyceridaemia) in three subjects in this group (subjects No. 1, 2, and 6) were judged as clinically significant; the increased triglyceride levels returned to their normal values one week later without any treatment. In the 800 mg TID cohort and placebo group, there were 14 and 5 AEs, reported, respectively. In the 800 mg Hydronidone group, adverse events included 3 instances each of liver dysfunction and hyperlipidemia, and one instance each of headache, dizziness, constipation, bloating, nausea, heartburn, skin rashes, and eosinophilia. In placebo group, adverse events included 2 instances of hyperlipidemia, 2 of diarrhea and 1 instance of abnormal liver function.

The values of low density lipoprotein cholesterol (LDL-CHOL) were slightly higher than the upper limit of normal (ULN) in two (2) subjects in each of the 800 mg TID Hydronidone dose group and placebo. These increases were considered as clinically nonsignificant and returned to their normal values upon cessation of treatment with the study drug. The AEs of abnormal liver function among the subjects in the 800 mg TID Hydronidone dose group included two cases of moderately but clinically significantly increased liver parameters. In one subject from this group, on the fifth day of dosing, the ALT and AST values were 79 U/L and 47 U/L, respectively. Upon repeated test on study day 6, their values were 101 U/L and 61 U/L, for ALT and AST, respectively, accompanied by heartburn, rash, headache and other symptoms. Due to progressive increase in liver enzymes, the Investigator terminated the subject due to perceived risk to subject's well-being. The subject was monitored in the following week. The transaminases returned to their normal, pre-dose values within a week after the study drug discontinuation, on its own, without treatment. Although this AE satisfied criteria for a SAE, it was classified by the Investigator as an important adverse event and not as SAE because the subject was prescribed no specific treatment.

In summary, the most common AEs associated with 10-day dosing with Hydronidone at 600 mg TID (total daily dose of 1800 mg) or 800 mg TID (total daily dose of 2400 mg) were hyperlipidemia (triglyceridaemia) and increased liver transaminases. These AEs were transient, resolving without treatment. Since they were observed both in the Hydronidone and placebo groups, it is unclear if these AEs were specifically related to the Hydronidone treatment.

### Example 7: PK Study of Single Ascending Oral Dose

This was randomized, open-label, 4-period, crossover PK study of single ascending oral doses of Hydronidone in healthy volunteers.

Ascending single oral doses of 200 mg, 400 mg, 600 mg and 800 mg of Hydronidone were given to 12 eligible, healthy male Chinese subjects, 26-38 years of age, satisfying all inclusion and exclusion criteria, and randomly divided into four dose groups, 3 subjects per group, under fasted conditions. According to the Williams Design, the subjects in each group were treated with Hydronidone at four different doses in four periods. The PK blood samples were collected at: 0 (pre-dose), 10 min, 20 min, 30 min, 40 min, 1 hr, 1.5 hr, 2 hr, 3 hr, 4 hr, 5 hr, 6 hr, 8 hr, 10 hr, 12 hr, 16 hr, and 24 hr, and 48 hr post-dose. The subjects also provided PK urine samples at 0 (pre-dose), 5 hr, 12 hr, and 24 hr post-dose. There was additional 24 hr washout period after the end of collection of PK samples, a total of 48 hrs between the first and the next dose level administration. Blood and urine samples were analysed for determination of plasma and urine concentrations of Hydronidone and its metabolites (M3).

TABLE 1
single oral dose PK parameters of hydronidone in healthy Chinese subjects (Example 7)
[Mean(SD)] in Study SG-L-200801

| | | 200 mg | 400 mg | 600 mg | 800 mg |
|---|---|---|---|---|---|
| PK parameter/Dose | Unit | (n = 12) | (n = 12) | (n = 12) | (n = 12) |
| AUCINE_pred | hr*mg/L | 1.38(0.37) | 2.8(0.76) | 5.01(1.42) | 6.31(1.45) |
| AUC_% Extrap_pred | | 1.63(1.45) | 0.59(0.36) | 1.71(3.29) | 0.358(0.54) |
| AUClast | hr*mg/L | 1.36(0.37) | 2.78(0.76) | 4.92(1.37) | 6.29(1.45) |
| Cl_F_pred | L/hr | 154(39.65) | 154(44.83) | 129(35.67) | 134(35.93) |
| Clr | L/hr | 0,881(0.66) | 0.668(0.21) | 0.59(0.25) | 0.522(0.29) |
| Cmax | ng/MI | 715(298.07) | 1400(467.57) | 2500(1003.5) | 2960(1107.49) |
| HL_Lambda_z | hr | 6.9(2.06) | 5.26(1.58) | 6.46(2.07) | 5.54(0.86) |
| Lambda_z | l/hr | 0.1093(0.03) | 0.1434(0.04) | 0.1141(0.02) | 0.1278(0.02) |
| Tmax | hr | 0.33(0.33-1.5*) | 0.33(0.33-1.5*) | 0.67(0.33-1.5*) | 0.67(0.33-2*) |
| Vz_F_pred | L | 1540(644.41) | 1180(530.69) | 1180(407.19) | 1080(336.89) |
| Amount_Recovered | L*mg/L | 1.27(1.34) | 1.92(0.87) | 2.86(1.19) | 3.22(2.03) |
| Percent_Recovered | % | 0.634(0.57) | 0.479(0.22) | 0.476(0.2) | 0.403(0.25) |

*For Tmax, Median (Min-Max)

The amount of intact drug excreted via urine during 48 h were 82.25±40.95 mg, 183.75±142.14 mg, 218.93±182.73 mg, and 234.72±210.50 mg. The urinary percentage of Hydronidone were 0.634±0.57%, 0.479±0.22%, 0.476±0.2%, 0.403±0.25%, which shows that most of the orally administered Hydronidone is not eliminated intact through the urine. The percentages of M3 excreted via urine were 29.47%, 32.94%, 26.14%, and 21.04%, showing that Hydronidone is transformed and subsequently excreted via urine in form of metabolites.

The administration of single oral doses of 200 mg, 400 mg, 600 mg and 800 mg of Hydronidone, the Chinese healthy subjects was safe and well tolerated with no adverse events nor SAEs reported.

### Example 8: Cross Over PK Study of Effects of Food on Safety, Tolerability, and PK of Single Oral does

This was a randomized, open-label, 2-period, crossover PK study of effects of food on safety, tolerability, and PK of single oral doses of Hydronidone in healthy Chinese subjects. The PK parameters of the M3 and M4 metabolites of Hydronidone, in presence or absence of food were also determined.

Twelve healthy Chinese subjects were enrolled, dosed and in this inpatient study. They were randomly assigned in two groups/periods to receive a single oral 600 mg (3×200 mg) dose of Hydronidone either under fasted, or fed (high fat) conditions. Within each treatment period, PK blood samples were collected at: 0 (pre-dose), 10 min, 20 min, 30 min, 40 min, 1 hr, 1.5 hr, 2 hr, 3 hr, 4 hr, 5 hr, 6 hr, 8 hr, 10 hr, 12 hr, 16 hr, and 24 hr, and 48 hr post-dose. The wash-out period between the two periods in this trial was 48 hours.

The PK results showed following the oral administration of a single 600 mg dose of Hydronidone, the presence of food (high-fat meal) delayed the rate of absorption of Hydronidone and its main metabolites M3 and M4, decreasing their Cmax values, however, without changing their exposures as shown by their unaffected AUC values. Therefore, and based on the results of AUC, it was indicated that taking food had no influence on the exposure level of Hydronidone and its major metabolites, M3 and M4. Hydronidone was safe and well tolerated, without SAEs or AEs reported.

### Example 9: Open-Label, Parallel Group PK Study of Multiple Ascending Oral Doses

This was randomized, open-label, parallel group PK study of multiple ascending oral doses of Hydronidone in healthy Chinese subjects.

In this study, ascending oral doses of Hydronidone of 200 mg, 400 mg, and 600 mg TID, were administered to 27 healthy subjects (15 males and 12 females) divided into 3 groups (9 subjects/group), for four (4) consecutive days. This was an inpatient study. Hydronidone was administered under fasted conditions. Blood and urine samples were collected for PK assessments. The PK assessments were performed upon single (after the first dose) and multiple dosing. Standard safety evaluations were also performed including physical examination, ECG, hematology, serum biochemistry and urinalysis.

The PK results showed that Hydronidone was rapidly absorbed with Tmax of approximately 1 hour. Upon single dosing, at 36 hrs. post-dose, both parent drug and major metabolites wee undetectable. The exposure levels of Hydronidone and metabolites from high to low in human plasma followed the rank order Hydronidone-M4>Hydronidone-M3>Hydronidone-M2; after the single Hydronidone administration, the total recovery of the intact drug and M2, M3, and M4 metabolites in the urine were 82.7%, 85.5%, and 84.0%, respectively. The PK results obtained from single and multiple dosing regimen indicated no obvious accumulation of Hydronidone. Male subjects had lower exposures as compared to female subjects across dose levels administered. The exposure was increasing with the dose but without strict dose proportionality due to high variability.

All 27 subjects completed the study. Hydronidone was safe and well tolerated in this study within the single dose range of 200-600 mg TID. No deaths nor SAEs were reported in this trial. There were seven (7) adverse events reported by two subjects from the 200 mg TID multiple-dose group and five (5) subjects from 400 mg TID multiple-dose group. The AEs reported in this part of the study included one instance each of dizziness, leukocytopenia, and abdominal pain, two instances each of abdominal distension and diarrhea. All AEs were deemed as probably related to Hydronidone treatment. The severity of the AEs was mild, all recovering on its own. All subjects with AEs completed the study.

### Example 10: Open-Label, Study of Safety, Tolerability and PK of Single Doses, 7-Day, and 4-Week Multiple Oral Doses

This was a randomized, open-label, study of safety, tolerability and PK of single doses, 7-day, and 4-week multiple oral doses of Hydronidone in healthy Chinese subjects.

This study was conducted in three parts. In the first part single escalating doses of Hydronidone of 15 mg, 30 mg, 60 mg, 90 mg, and 120 mg/day were administered to six (6), eight (8), eight (8), six (6), six (6) healthy subjects, respectively, as per the Fibonacci Escalating Method (modified Fibonacci method). The age, height, weight, body mass index and gender composition ratio of subjects in each dose group were comparable at baseline. As no adverse experiences were reported at any dose level, the two highest single dose levels of 90 mg and 120 mg of Hydronidone were selected for the second part of the study, and were administered TID to 6 subjects each, for seven consecutive days. Thus, total daily doses of Hydronidone in these two dose groups were 270 mg and 360 mg, respectively. All subjects completed the seven-day dosing regimen without premature discontinuation. No clinically-significant abnormal changes in vital signs, ECG and laboratory test results of each group of subjects were found. In the third part of the study, six (6) subjects were given oral Hydronidone at a daily dose of 60 mg TID (total daily dose of 180 mg) for four (4) consecutive weeks. All subjects completed the seven-day dosing regimen without premature discontinuation. No clinically-significant abnormal changes in vital signs, ECG and laboratory test results of each group of subjects were found.

PK assessments were performed in each part of the study. In the single part of the study, the PK parameters also included food effect assessment.

In the single dose part of the study (Part I), the main pharmacokinetic parameters of Hydronidone derived from 36 healthy subjects who were administered orally single doses of 30, 60, and 120 mg of Hydronidone in fasting state were as follows: Tmax was (0.60±0.49).

Hydronidone single doses in the range of 15-120 mg were safe and well tolerated by all subjects in the study. All subjects completed all study procedures and observations. No deaths, SAEs or AEs occurred throughout the study, and no abnormal change in subjects' subjectively perceived symptoms, and physical examination were observed after administration. There were no abnormalities in any of the vital signs post-dose at any time point, as compared to baseline and between different dose groups (p>0.05). Three subjects experienced an increase in their T-BIL and D-BIL post-dose values, two of which were in the 30 mg dose group, and one in the 120 mg dose group. Upon re-test, these values were within their normal range. None of these increases were deemed as clinically significant by the Investigator.

Hydronidone dosing for 7 consecutive days at 90 mg TID (270 mg/day) and 120 mg TID (360 mg/day) was safe and well tolerated by all subjects in the study. There were no premature discontinuations and all subjects completed the study. No deaths, SAEs or AEs occurred throughout the study, and no abnormal change in subjects' subjectively perceived symptoms, and physical examination were observed after administration. No clinically-significant abnormal changes in vital signs, laboratory parameters, and ECG parameters were found in any dose groups (p>0.05).

Hydronidone dosing for 4 consecutive weeks at 60 mg TID (180 mg/day) was safe and well tolerated by all subjects in the study. There were no premature discontinuations and all subjects completed the study. No deaths, nor SAEs were reported.

With regard to AEs, there were no AEs reported in the tolerability part of the study; there were only two instances of mild transient dizziness that occurred in two female subjects (out of 12 subjects) 30 min and 1.0. hr following the dosing in the PK group dosed with multiple doses of 60 mg TID of Hydronidone. These AEs resolved without treatment within 1-1.5 hr.

No clinically-significant abnormal changes in vital signs, and ECG parameters were found in any dose groups (p>0.05). Observed variations in laboratory parameters were deemed as clinically non-significant by the Investigator. There was no abnormal prolongation of the QTc interval at any dose level.

In conclusion, Hydronidone was safe and well tolerated in a range of single oral doses from 15 mg to 120 mg. The maximum tolerated single oral dose of Hydronidone was 120 mg. Hydronidone was also safe and well tolerated up to 360 mg/day when administered as 120 mg TID for seven consecutive days. The Hydronidone was safe and well tolerated at the dose of 180 mg/day when administered as 60 mg TID to healthy subjects for four (4) consecutive weeks. There was no accumulation of the drug in the body as indicated by the PK analysis of the main PK parameters at comparable dose levels of Hydronidone following single and multiple dosing regimen.

### Example 11: Phase II Clinical Pharmacology Studies of Hydronidone

This is a Phase II, randomized, double-blind, placebo-controlled, Entecavir-Basic Treatment, multi-center, dose-ranging study on the efficacy and safety of Hydronidone when administered to Chinese subjects with liver fibrosis due to chronic Hepatitis B for 52 consecutive weeks.

The main objective of this study is to explore the effective doses and assess the safety of Hydronidone when administered concomitantly with anti-viral drug Entecavir to Chinese subjects with liver fibrosis due to chronic hepatitis B infection for 52 consecutive weeks. The second objectives of this study are to evaluate the effects of Hydronidone on improvement of liver inflammation and liver function when administered concomitantly with anti-viral drug entecavir to Chinese subjects with liver fibrosis due to chronic hepatitis B infection.

Approximately 240 eligible subjects, approximately 60 subjects per dose cohort, are planned to be enrolled in the study and treated daily with Hydronidone oral doses of 30 mg, 60 mg. or 120 mg TID (total daily doses of 180 mg, 270 mg, and 360 mg, respectively) for 52 consecutive weeks or placebo, concomitantly with basic anti-viral treatment with Entecavir at a daily dose of 0.5 mg. In parallel, a group of 12 subjects will be evaluated for PK.

### Example 12: Ishak Score Analysis of Drug Groups (Primary Endpoint)

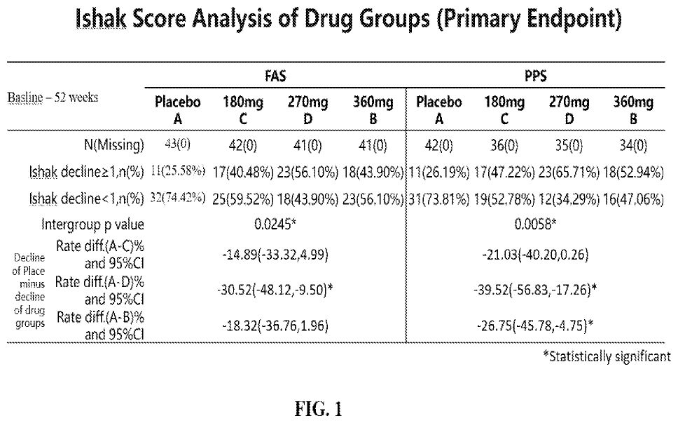

As shown in FIG. 1, After 52 weeks of therapy, hydronidone was able to significantly lower the Ishak score: ≥1.270 mg group appears to be the best.

### Example 13: Analysis of Intra Group Ishak Score Change

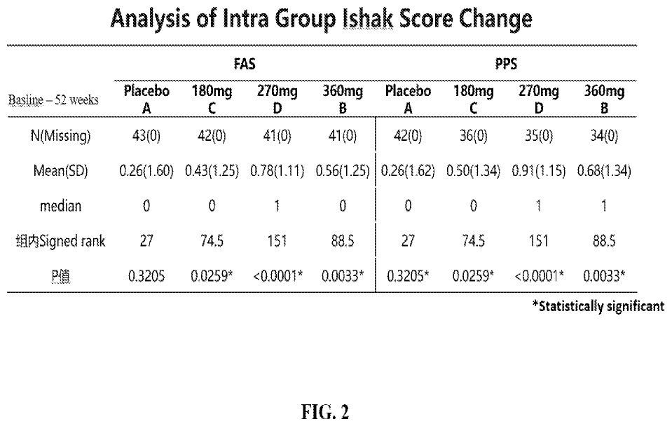

As shown in FIG. 2, after 52 weeks of therapy, all drug groups showed statistically significant decline of Ishak score≥1. Placebo group showed no statistical decline of Ishak score≥1.

### Example 14: Analysis of Patients of Significant Fibrosis

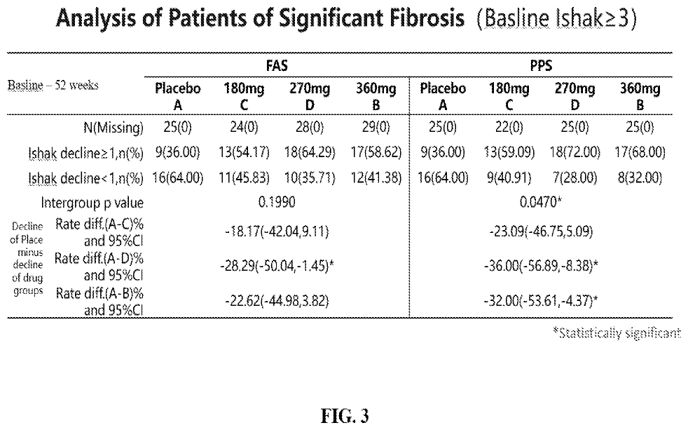

As shown in FIG. 3, Ishak score improvement is consistent with the results of primary endpoint analysis. 270 mg group appears to the be the best dose group.

### Example 15: Combination Analysis of 270 mg and 360 mg Groups

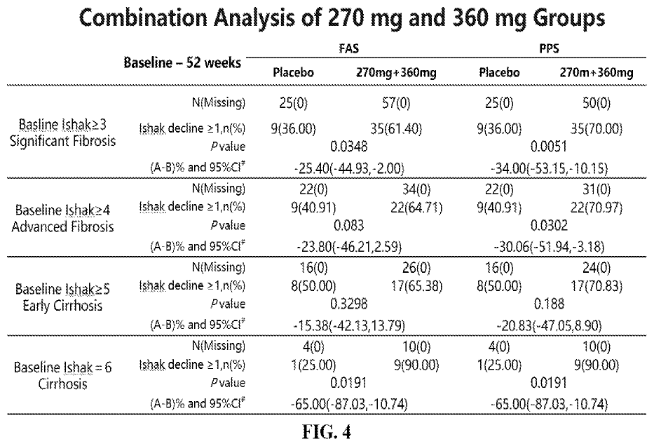

As shown in FIG. 4, hydronidone can improve fibrosis in patients of significant fibrosis and cirrhosis

### Example 16: Combination Analysis of All Drug Groups

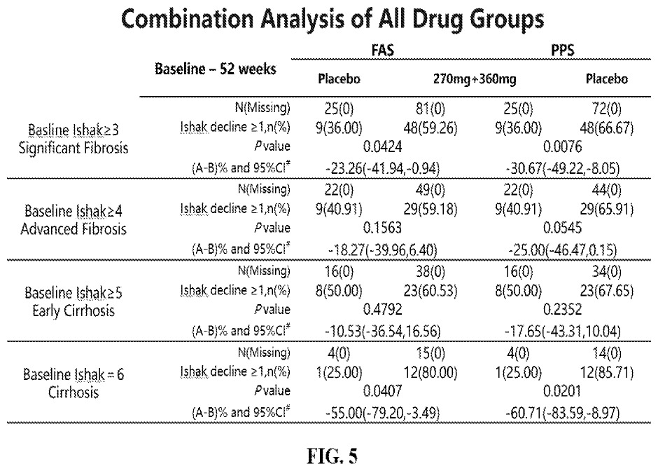

As shown in FIG. 5, hydronidone can improve fibrosis in patients of significant fibrosis and cirrhosis.

### Example 17: Ishak Score Changes of Baseline HBeAg (+) Patients after 52 Weeks

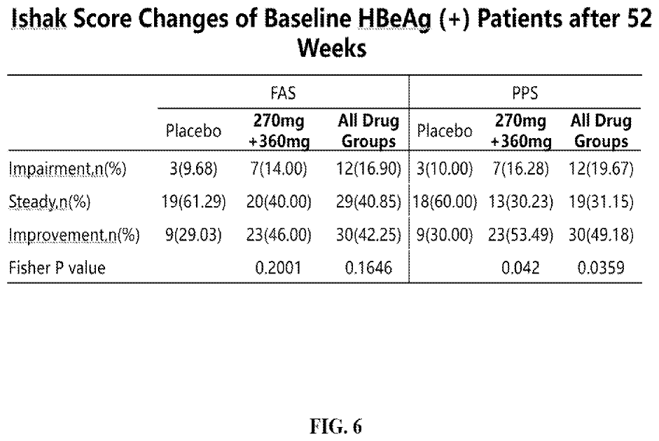

As shown in FIG. 6, hydronidone showed statistical significant changes when dose groups are combined.

### Example 18: Ishak Score Changes of Baseline HBeAg (−) Patients after 52 Weeks

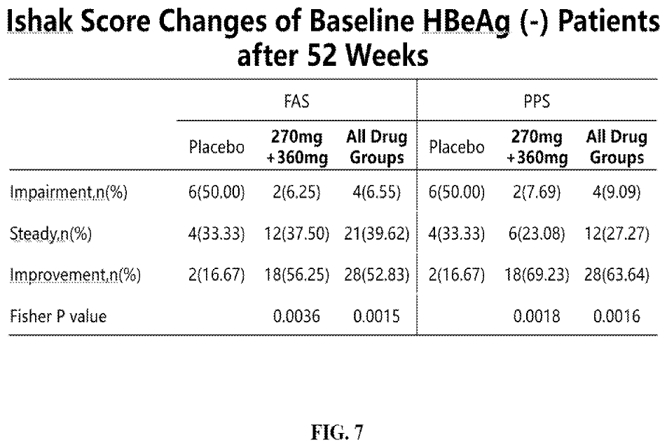

As shown in FIG. 7, Hydronidone showed statistical significant changes when dose groups are combined. Improvement in HBeAg(−) patients appears to be better than in HbeAg(+) patients.

### Example 19: Change of Liver Stiffness Measurement LSM

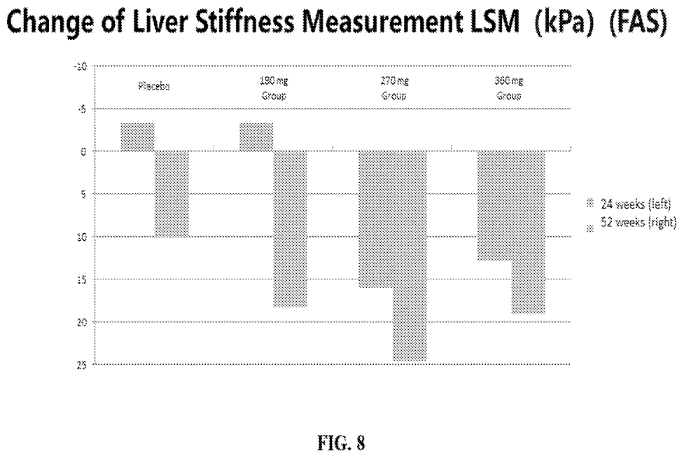

As shown in FIG. 8, after 52 weeks of therapy, all drug groups show decline of LSM(kPa) (intra group P<0.01).

### Example 20: Combination of Drug Groups for LSM Analysis

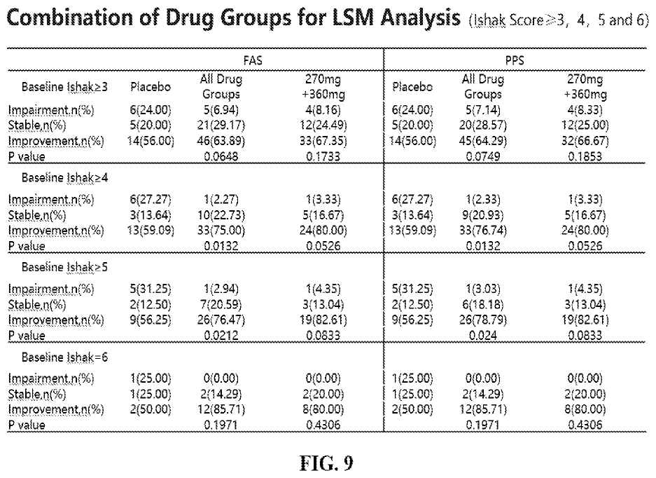

As shown in FIG. 9, in Advanced Fibrosis (Ishak≥4), LSM scores showed statistical significance.

### Example 21: Analysis of HBV DNA+ Patients after 52 Weeks Entecavir Therapy

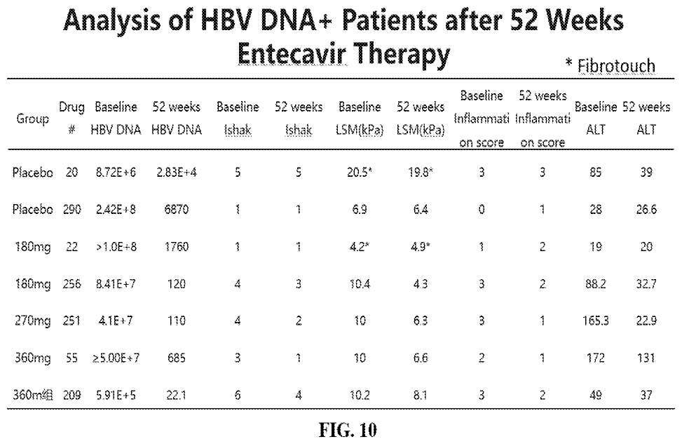

As shown in FIG. 10, all patients (Ishak>3) responded to hydronidone therapy.

### Example 22: Effect of Hydronidone on (CCl4+HFD) Induced NASH Model in Mice

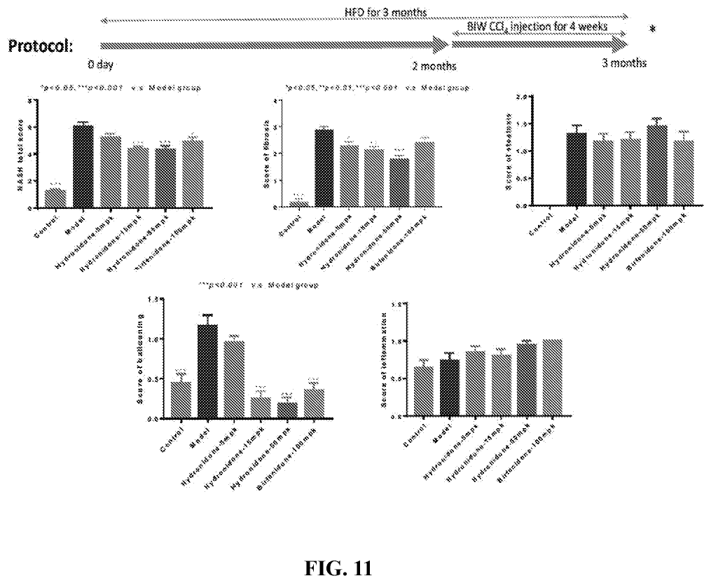

As shown in FIG. 11, a variety of tests were performed to illustrate the use of hydronidone at different dosages. The total NASH score was lower at dosage of 15 milligram per kilogram (mpk) and 50 mpk.

### Example 23: H&E Staining Using Hydronidone

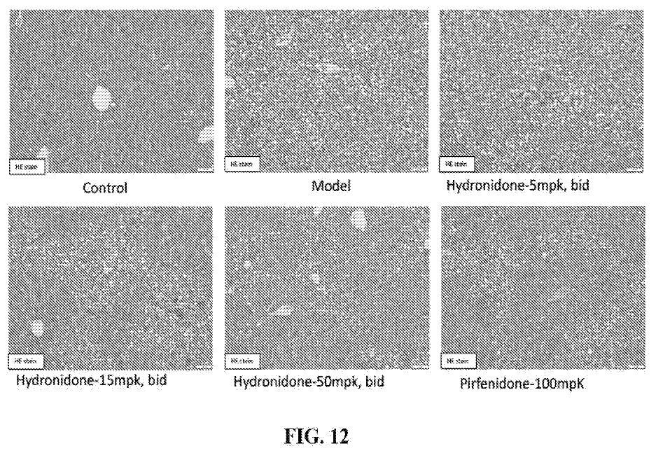

As shown in FIG. 12, hydronidone at different dosages were used to show the effect by H&E staining. Hydronidone has a protective effect on CCl₄ and western diet (WD) induced NASH. Hydronidone at 15 mpk and 50 mpk significantly inhibited CCl₄ and WE induced fibrosis and cell ballooning. Hydronidone is more potent than pirfenidone.

1-25. (canceled)

26. A pharmaceutical composition comprising about 20% to about 90% weight percentage of hydronidone or a salt thereof and one or more pharmaceutical acceptable excipients selected from consisting of magnesium stearate, calcium carbonate, zinc stearate, calcium stearate, stearic acid, palmitic acid, myristic acid, and glyceryl dibehenate.

27. The pharmaceutical composition of claim 26, wherein said weight percentage of hydronidone or a salt thereof is about 25% to about 35%.

28. The pharmaceutical composition of claim 26, wherein said weight percentage of hydronidone or a salt thereof is about 30%.

29. The pharmaceutical composition of claim 28, wherein said one or more pharmaceutical acceptable excipients comprise magnesium stearate.

30. A method of treating a subject suspected of having or being at risk of developing nonalcoholic steatohepatitis (NASH) comprising administering to the subject a therapeutically effective amount of the pharmaceutical composition of claim 26.

31. A method of treating a subject comprising identifying a subject having a liver stiffness measurement value of at least 4 kiloPascals (kPa) and administering to the subject a therapeutically effective amount of the pharmaceutical composition of claim 26.

32. The method of claim 31, wherein the liver stiffness measurement value is from about 4 kPA to 13 kPa.

33. The method of claim 31, wherein the liver stiffness measurement value is at most 75 kPa.

34. A method of treating a subject comprising identifying a subject with an Ishak value that is from about 1 to 6; and administering to the subject a therapeutically effective amount of the pharmaceutical composition of claim 26.

35. The method of claim 34, wherein said Ishak value is from about 1 to 3.

36. The method of claim 34, wherein said Ishak value is from about 3 to 6.

37. A method of treating a subject comprising identifying a subject that is substantially free of hepatitis B viral protein; and administering to a subject a therapeutically effective amount of the pharmaceutical composition of claim 26.

38. A method of treating a subject comprising identifying a subject wherein the subject tests positive for hepatitis virus DNA after receiving one or more courses of anti-viral treatment; and administering to the subject a therapeutically effective amount of the pharmaceutical composition of claim 26.

39. The method of claim 38, wherein the hepatitis virus DNA is selected from hepatitis A virus, hepatitis B virus, hepatitis C virus, hepatitis D virus, and hepatitis E virus.

40. The method of claim 30, wherein said weight percentage of hydronidone or a salt thereof is about 30%.

41. The method of claim 30, wherein said pharmaceutical excipients comprise magnesium stearate.
